(19) United States
(12) Patent Application Publication
(10) Pub. No.: US 2010/0068720 A1
(43) Pub. Date: Mar. 18, 2010

(54) Method and kit for detection of early cancer or pre-cancer using blood and body fluids

(76) Inventors: Weiwei Li, Lindenhurst, NY (US); Jessica Li, Lindenhurst, NY (US)

Correspondence Address: Weiwei. Li, 338 38th Street, Lindenhurst, NY, 11757, US

(21) Appl. No.: 12/583,472

(22) Filed: Aug. 21, 2009

Related U.S. Application Data
(63) Continuation of application No. 11/303,815, filed on Dec. 15, 2005.

(51) Int. Cl. C12Q 1/68

(52) U.S. Cl. 435/6

(57) ABSTRACT

This invention is related to a method for the detection of early or pre-cancer using DNA isolated from blood and body fluids. This invention provides an improved methylation-based PCR assay, a panel of methylated-based cancer-related gene markers for the detection of general cancer and a panel of unmethylation-based tissue- or cell-specific gene markers for discriminating different type of cancer detected from blood samples. This method couples a sequential cancer-related gene marker detection and tissue or cell-specific gene marker assay and is particularly useful as a simultaneous screening test for following type of cancer: lung, breast, ovarian, colon, stomach, prostatic, pancreatic and liver cancer.

This is a continuation of parent application Ser. No. 11/303,815 filed on Dec. 8, 2005 and entitled “Method and kit for detection of early cancer or pre-cancer using blood and body fluids”

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

1. Field of the Invention

This invention is related to a method for the detection of early or pre-cancer using DNA isolated from blood and body fluids. This invention provides an improved methylation-based PCR assay, a panel of methylation-based cancer-related gene markers for the detection of general cancer and a panel of -based tissue- or cell-specific gene markers for discriminating different type of cancer detected from blood samples. The cancer type discriminated or localized can be further confirmed by assaying the panel of methylated-based cancer-related gene markers using a sample from corresponding body fluid. This method couples a sequential cancer-related gene marker detection and tissue or cell-specific gene marker assay and is particularly useful as a simultaneous screening test for following type of cancer: lung, breast, ovarian, colon, stomach, prostatic, pancreatic, liver and nasopharyngeal cancer.

2. Description of the Related Art

Detection of cancer at an early stage is critical for successful treatment and increasing survivability. Cancer arises due to the accumulation of DNA alterations that results in cells to uncontrollably proliferate. The most common DNA alteration (>95%) is epigenomic-caused methylation in the promoter of some specific genes such as tumor suppression genes. Methylation of the specific genes is demonstrated to be early and stable molecular fingerprints of pre-cancer and cancer cells. Methylation of CpG islands involves the course in which DNA methyltransferases (Dnmts) transfer a methyl group from S-adenosyl-L-methionine to the fifth carbon position of the cytosines. It was well demonstrated that methylation patterns of DNA from cancer cells are significantly different from those of normal cells. DNA of cancer cells is generally hypomethylated compared to that of normal cells (Feinberg et al, Nature, 301: 89-92, 1983; Gama-Sosa et al, Nucleic Acids Res, 1: 6883-689494, 1983). However DNA of cancer cells could be more methylated than that of normal cells in the selected regions such as in the promoter regions of tumor suppressor genes. Thus, the detection of the methylation patterns or ratio in the specific genes could lead to the discrimination of cancer cells from normal cells, thereby indicating the presence of pre-cancer or early cancer.

More than 6000 published research articles described the relationship between cancer and gene methylation, which established a solid basis for gene methylation-characterized cancerous change. There have been many methods for the detection of DNA methylation. The most widely used method among these is methylation-specific PCR (MS-PCR). This assay, through chemical modification of DNA, selectively amplify methylated sequences with primers specific for methylation (Herman et al., Proc. Natl. Acad. Sci. USA 93: 9821-9826 (1996). After PCR a gel-based detection is processed. MS-PCR was recently improved by using real-time probe system. These improved methods such as MethyLight, Q-MSP, and HM-MethyLight showed to be more sensitive, specific, quantitative and high throughput than original MS-PCR (Eads et al., Cancer Res, 59: 2302-2306, 1999; Cottrell et al., Nucleic Acid Res, 32: e10, 2004). However, all existing methylation technologies are still not sufficient to apply to routine cancer detection, even including MethyLight, a method considered to be potential for clinical application. A critical weakness of these existing methods is that their clinical sensitivity is still too low when a sample from non-invasive approach such as from plasma/serum or other remote media is used. It was demonstrated that a cancer at its early stage may release its cells or free DNA into blood or body fluids through cell detachment, vascularization or apoptosis, respectively (Holdenrieder et al Ann NY Acad Sci, 945: 93-102, 2001; Jahr et al, Cancer Res, 61: 1659-1665, 2001). Free DNA from cancer in circulation establishes a basis for cancer detection using DNA methylation technology. However the quantity of DNA or cells released into blood or body fluids from early stage of tumor is quite low, which obviously limits successful methylation-based PCR assay, especially when a panel of genes is examined. Another possibility for low sensitivity of current methylation-based PCR assay is that an appropriate panel of methylation-based biomarkers is not yet established. Most studies of methylation marker assay focused on either 2-3 known genes or on the global analysis of unknown CpG islands, which are not suitable for practical use for cancer detection. At least 50 methylation-based biomarkers were found so far. Several panels of biomarker selected from these DNA methylation biomarkers have been tested using the blood or body fluid samples (Laird et al, Nature reviews, 3: 253-266, 2003). However these testing could not show that the selected biomarkers are really suitable for early detection of cancer as the low frequency of detection. Furthermore, DNA methylation biomarkers found and used by current assay are only suitable for detection of general cancer. It was well demonstrated that methylation of a single gene as a biomarker can occur in different type of cancers and a special type of cancer can have methylation of numerous genes. Therefore there is ample need for improvement of methylation-based detection of early cancer by using blood and body fluids. This improvement could involve to (1) increase sensitivity of assay by increasing amount of modified DNA available for PCR assay and by improving PCR assay condition; (2) establish an appropriate panel of methylation-based biomarkers which should have a high frequency occurred in most of common cancer; and (3) establish a panel of tissue- or cell-original biomarkers which could be detected in blood by using same methylation-based assay and could be used for discriminating different type of cancer.

### BRIEF SUMMARY OF THE INVENTION

The present invention provides an improved methylation-based PCR assay, a panel of methylation-based cancer-related gene markers for detecting the presence of early or pre-cancer using DNA from blood or body fluids. This invention also provides a panel of unmethylation-based tissue- or cell-specific gene markers to localize the source of DNA so that discrimination among different types of cancer is able to make. This invention further couples a cancer-related gene marker detection and tissue or cell-specific gene marker assay and is particularly useful as a simultaneous screening test for following types of cancer: lung, breast, ovarian, colon, stomach, pancreatic, liver, prostate and nasopharyngeal cancer.

The method of this invention includes analyzing plasma/serum samples first for methylation of a panel of genes related to tumor suppressor, cell cycle and DNA repair. Methylation of these genes is indicative of the presence of an early cancer or pre-cancer. The method of this invention also includes analyzing next for unmethylation of a panel of genes related to tissue- or cell-specific gene activation, which is indicative of tissue or cellular source of DNA. In certain embodiments, the method of this invention can include analyzing other body fluid samples such as sputum and urine for methylation of a panel of genes related to tumor suppressor, cell cycle and DNA repair to confirm the findings of using blood samples. The method of this invention can be carried out as a routine clinical assay on a subject having suspected cancer.

The present invention can provides a method for health management of a subject. According to the method of this invention, a subject detected to have suspected cancer by the first analysis of gene methylation can be subject to the second analysis of tissue origin of DNA to determine type of a cancer. A traditional diagnostic method such as CT or MRI or colonoscopy could be then carried out, depending on site, to accurately confirm the presence of a cancer. The present invention also provides a method for monitoring cancer progressing of a subject by analyzing plasma/serum samples for recurred or increased methylation of a panel of genes. Cancer progressing includes therapeutic effectiveness, recurrence and metastasis of a cancer.

The present invention further provides a kit for detecting the presence of an early cancer or pre-cancer and determining the type of the cancer. The kit includes a group of primer pairs, a group of oligonucleotide probes that are labeled with different dye and are preferably conjugated with minor groove binder. The kit can also include a DNA modification buffer and PCR reaction buffer for convenient use.

### DESCRIPTION OF THE FIGURES

FIG. 1 shows a diagram of detecting early cancer or pre-cancer by coupling methylated gene marker detection and tissue- or cell-specific gene marker assay. The process involves to (1) the collection of blood or body fluids; (2) isolation and modification of DNA; (3) gene amplification analysis of the first panel of methylation-based, cancer-related gene markers to indicate if a cancer or pre-cancer exists, and (4) gene amplification analysis of the second panel of unmethylation-based, tissue- or cell-specific gene markers to indicate the tissue or cellular source of DNA.

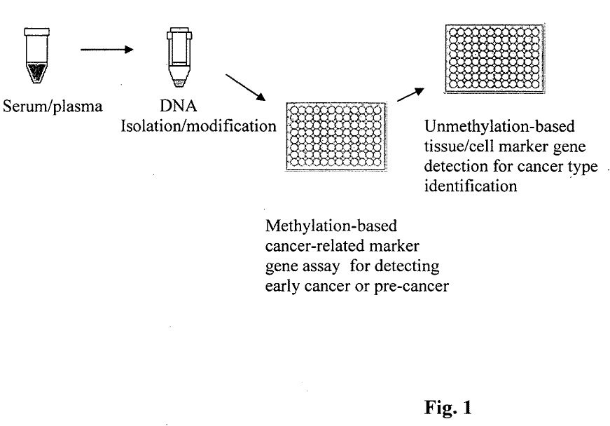

FIG. 2 shows the yield of modified circulating DNA from plasma by using the method of this invention. The experiment was carried out as described in example 1.

FIG. 3 shows methylated gene marker analysis of blood samples using multiplexed real-time PCR based on this invention. The experiment was carried out as described in example 2.

### DETAILED DESCRIPTION OF THE INVENTION

The present invention provides a new, rapid method for detection of early cancer or pre-cancer, as well as a method for discriminating different type of cancer using a DNA methylation-based PCR assay. The present invention also provides a kit which could be particularly useful for a route clinical assay in the detection of general cancer and determination of special type of cancer.

According to the method of this invention, a biological sample can be from whole blood, plasma, serum and other body fluids that include cerebro-spinal fluid, tears, sweat, lymph fluid, saliva, nasal swab or nasal aspirate, sputum, bronchoalveolar lavage, breast aspirate, pleural effusion, peritoneal fluid, glandular fluid, amniotic fluid, cervical swab or virginal fluid, ejaculate, semen, prostate fluid, urine, conjunctival fluid, duodenal juice, pancreatic juice, bile and stool. If a plasma sample is used in this invention, the sample should be obtained under the appropriate condition. For example, anti-coagulants contained in whole blood should be able to inhibit DNAse activity. A suitable anti-coagulant may be a chelating agent such as ethylenediaminetetraacetic acid (EDTA) that prevents both DNAse-caused DNA degradation and clotting of the whole blood samples. If other body fluid sample such as sputum is used, Cells in this kind of samples can be collected by the procedures described in prior art. For example, collection of cells in a urine sample can be achieved by simply centrifugation, while collection of cells in a sputum sample requires DTT treatment of sputum followed by filter through nylon gauze mesh filter and centrifugation. Genomic DNA can be isolated and chemically modified by standard methods or kits commercially available. More preferably, DNA is isolated and chemically modified by a coupled DNA isolation and modification method. This method is particularly useful for gaining a high yield of modified DNA from a small quantity of starting materials. This method is also particularly useful for fast isolation and modification of DNA in a short time.

The method uses the high concentrations of non-chaotropic salts such sodium chloride in association with protein enzyme inhibitors to isolate DNA from plasma, serum and other body fluids. A high concentration of non-chaotropic salts is able to cause the dissociation of proteins from DNA and protein molecules precipitation from solution, and further enables DNA to bind to a solid matrix such as silica dioxide. DNA is captured with an apparatus comprising a column pre-inserted with a silica gel, or a silica membrane, or a silica filter. According to this method that a column is a micro-spin column which fits a 1.5 or 2.0 ml micro-centrifuge tube, and the combination of the column and the micro-centrifuge tube further fits inside a table-top microcentrifuge. The DNA-bound silica matrix is washed by adding a washing buffer and eluted with TE buffer or water.

The DNA isolated in this manner is able to be directly used for chemical modification. According to this method the composition of modifying reagents comprise a bisulfite, a potassium salt, or a magnesium salt, and an EDTA. The composition of modifying reagents is made in a solution form. An advantage of the composition according to this method is that unmethylated cytosine residues can be maximally converted to uracil in a single-stranded DNA, while methylated cytosine remains unchanged. Another advantage of the composition according to this method is that degradation of DNA resulted from chemical, biochemical and thermophilic action in modification is efficiently prevented or reduced. A further advantage of the composition according to this method is that the DNA modification process is much shorter without interrupting a completed conversion of unmethylated cytosine to uracil and without a significant thermodegradation of DNA resulted from depurination.

Once DNA modification is complete, DNA is captured, desulphonated and cleaned. The modified DNA can be captured by a solid matrix such as silica dioxide, in the presence of high concentrations of non-chaotropic salts. It is preferred according to this method that modified DNA is captured with an apparatus comprising a column pre-inserted with a silica gel, or a silica membrane or a silica filter. It is further preferred according to this method that a column is a micro-spin column which fits a 1.5 or 2.0 ml micro-centrifuge tube, and the combination of the column and the micro-centrifuge tube further fits inside a table-top microcentrifuge. After the modified DNA is applied to the column, a binding buffer consisting of non-chaotropic salts can be added to further enhance the binding of the modified DNA to silica matrix. The DNA-bound silica matrix is washed by adding a washing buffer. The modified DNA is further desulphonated on the column with an alkalized solution. Once desulphonation of modified DNA bound to silica matrix has been completed, the column is further washed with the washing buffer 2-3 times. The modified DNA is then eluted from the column and collected into a capped microcentrifuge tube. An elution solution could be DEPC-treated water or TE buffer (10 mM Tris-HCL, pH 8.0 and 1 mM EDTA). Both quality and quantity of eluted modified DNA can be measured by conventional techniques such as pico-green DNA measurement or by PCR amplification. By using the method, the required amount of plasma or serum for an assay point of gene methylation may be as low as 40 ul (assuming the minimum DNA amount in plasma/serum is 0.5 ng/ml). The required number of cells from other body fluids or small in vitro culture may be as few as 5 cells for an assay point of gene methylation.

Such modified DNA can be used for cancer-related gene methylation determination. According to the method of this invention, a panel of genes whose methylation is used for biomarkers of early cancer is comprised of the genes that increase DNA repair, decrease DNA damage, inhibit cell cycle, and function as tumor suppressor. Hypermethylation of these genes in the regulatory regions such as the promoter or promoter/exon 1 results in lose of the function of these genes. Functional lose of these genes has been shown to be a critical cause in mutagenesis, tumorigenesis, cancer development and spread. These genes mainly include cyclin-dependent kinase inhibitor 2A (p16), adenomatous polyposis coli (APC), glutathione S-transferase (GSTP1), Ras association domain family 1 (RASSF1A), human mutL homolog 1 (hMLH-1), O6-methylguanine-DNA methyltransferase (MGMT), E-cadherin (CDH1), H-cadherin (CDH13), death associated protein kinase (DAPK), runt-related transcription factor 3 (RUNX3), and Retinoic acid receptor beta (RAR-beta). According to prior art, the estimated methylation frequency of these genes in order in different type of cancer is listed as follow:

Lung cancer: p16-APC-RASSF1A-RUNX3-CDH1-CDH13-DAPK-MGMT-hMLH1-GSTP1-RARbeta;
Colon-rectal cancer: CDH13-p16-hMLH1-MGMT-CDH1-RASSF1A-APC-RARbeta-RUNX3-DAPK-GSTP1
Pancreatic cancer: RASSF1A-CDH13-p16-MGMT-RARbeta-hMLH1-APC-GSTP1-DAPK-RUNX3-CDH1
Liver cancer: RASSF1A-p16-GSTP1-MGMT-hMLH1-APC-RUNX3-DAPK-CDH1-RARbeta-CDH13
Stomach cancer: p16-APC-RASSF1A-MGMT-DAPK-hMLH1-RUNX3-CDH1-RARbeta-CDH13-GSTP1
Breast cancer: RASSF1A-APC-p16-CDH1-GSTP1-CDH13-hMLH1-RARbeta-MGMT-DAPK-RUNX3
Ovarian cancer: RASSF1A-p16-APC-hMLH1-GSTP1-CDH13-CDH1-MGMT-RARbeta-DAPK-RUNX3
Endometrial cancer: hMLH1-APC-p16-RASSF1A-CDH1-CDH13-GSTP1-DAPK-MGMT-RARbeta-RUNX3
Cervical cancer: CDH13-p16-APC-DAPK-RASSF1A-CDH1-RARbeta-MGMT-GSTP1-hMLH1-RUNX3
Prostate cancer: GSTP1-RASSF1A-RARbeta-APC-MGMT-p16-DAPK-hMLH1-CDH13-CDH1-RUNX3
Kidney cancer: RASSF1A-p16-GSTP1-hMLH1-APC-MRMT-RARbeta-DAPK-CDH1-CDH13-RUNX3
Urinary bladder cancer: RASSF1A-RARbeta-CDH1-CDH13-DAPK-APC-GSTP1-p16-hMLH1-MGMT-RUNX3
Esophagus cancer: RASSF1A-RARbeta-APC-MGMT-CDH1-p16-CDH13-GSTP1-hMLH1-DAPK-RUNX3
Nasopharygeal Cancer: RASSF1A-p16-RARbeta-CDH1-DAPK-MGMT-GSTP1-hMLH1-APC-CDH13-RUNX3
Lymphoma: p16-DAPK-RASSF1A-MGMT-hMLH1-RARbeta-GSTP1-APC-CDH1-CDH13-RUNX3

### Leukemia: MGMT-RARbeta-DAPK-p16-CDH13-CDH1-APC-RASSF1A-hMLH1-GSTP1-RUNX3

Therefore, more practically, according to the method of this invention, a panel of marker genes is comprised of p16, RASSF1A, APC, MGMT, hMLH1, GSTP1 and CDH-13. P16 plays a critical role in blocking progression through G1 phase of cell cycle by inhibition cyclin-dependent kinase 4. P16 is also well-known to inhibit tumorigenesis as a tumor suppression gene. RASSF1A, APC and CDH 13 also play an important role in inhibiting tumorigenesis and tumor expansion and are considered as the tumor suppression genes. MGMT and hMLH1, as genes encoding DNA repair enzymes, may be responsible for inhibiting gene mutation and removing genetic lesion from normal cells. One advantage of using this panel of genes for early cancer detection is that it is suitable for high throughput format of routine use i.e.: for 96 well PCR plate; another advantage of using this panel of genes is that methylation of these genes frequently occurs in cancer but rare or not occurs in normal tissue when combined together and used as a panel of genes; another advantage is that methylation of each of these genes can occur in most kind of cancer or is very specific to some type of cancer. Further advantage is that methylation of these genes is easy to be detected from plasma/serum or body fluid samples.

Table 1 shows the probability (% sensitivity) for detected methylation of a gene or more from the panel of marker genes selected according to the method of this invention.

TABLE 1
Estimated un-methylated probability of genes used for early cancer
detection

| | | % unmethylated | | | | | | |
|---|---|---|---|---|---|---|---|---|
| Type | % sensitivity | P16 | RASSF1A | APC | MGMT | GSTP1 | CDH13 | hMLH1 |
| Lung | 96 | 39 | 66 | 53 | 82 | 95 | 60 | 60 |
| Breast | 80 | 85 | 70 | 75 | 100 | 85 | 70 | 75 |
| Colon | 86 | 75 | 80 | 90 | 80 | 98 | 50 | 60 |
| Stomach | 87 | 48 | 75 | 80 | 90 | 85 | 100 | 60 |
| Liver | 93 | 45 | 48 | 80 | 85 | 65 | 80 | 98 |
| Pancreatic | 84 | 70 | 50 | 85 | 70 | 100 | 90 | 85 |
| Cervical | 85 | 70 | 85 | 75 | 90 | 80 | 60 | 80 |
| Esophagus | 84 | 80 | 70 | 67 | 70 | 98 | 68 | 92 |
| Prostate | 88 | 75 | 73 | 86 | 90 | 45 | 70 | 90 |
| Lymphoma | 86 | 27 | 90 | 100 | 80 | 90 | 90 | 90 |
| Subtotal | 86.9 | | | | | | | |
| Nasopharyn | 76 | 70 | 60 | 100 | 80 | 90 | 100 | 80 |
| Ovarian | 68 | 80 | 70 | 90 | 100 | 100 | 70 | 90 |
| Kedney | 65 | 90 | 60 | 86 | 95 | 90 | 88 | 100 |
| Bladder | 80 | 80 | 70 | 68 | 95 | 95 | 60 | 95 |
| Endometrial | 80 | 80 | 60 | 80 | 100 | 100 | 60 | 90 |
| Leukemia | 67 | 80 | 90 | 100 | 70 | 100 | 70 | 95 |
| Total | 81.6 | | | | | | | |

The methods for gene methylation determination may include but are not limited to MS-PCR, Primer extension, COBRA, MethyLight, ConLight-MSP, McMSP, MSP/DHPLC, SNuPE, HM-MethyLight, strand displacement assay, ligase chain reaction, rolling circle amplification, loop-mediated amplification, Oligonucleotide microarrays, Bisulfite sequencing and PyroMeth. However, a suitable method for routine clinical use should be rapid, cost-effective enough with high sensitivity and specificity. According to this invention, the method for cancer-related gene methylation determination is related to multiplex real-time gene methylation amplification. According to this method, the CpG-rich regions in the promoter or exon 1 of a gene marker can be amplified using at least two primers and a probe which are complementary to a methylated DNA converted with bisulfite. The primers are specific to methylated DNA of interest and are able to initiate synthesis of a primer extension product. A sequence-specific probe is able to hybridize to the target between forward and reverse primer sites. The probe is labeled with a fluorescent dye at 5′ portion. The fluorescent dyes may include but are not limited to FAM, TET, Texas-red, cy3, cy5, JOE, HEX, MAX, ROX, TAMRA and VIC. The probe is quenched with a black hole quencher and conjugated with a minor groove binder (MGB) protein at its 3′ portion. The MGB is able to fold into minor groove of DNA duplex created between the target sequence and probe. Thus the conjugation of MGB to the probe stabilizes annealing, allowing increased target discrimination, greater precision and consistence between individual assays. The probe used in the method of this invention can be also a Locked Nucleic Acid (LNA) probe which contains nucleosides having a methylene bridge that connects the 2′-oxygen of ribose with the 4′-carbon of the ribose ring. The LNA probe is also labeled with a fluorescent dye at 5′ portion and quenched with a black hole quencher at its 3′ portion. The LNA fluorescent probe may also increase thermal stability and hybridization specificity. Two or more gene markers can be multiplexed in a single amplification reaction to save time and reagents. A housekeeping gene such as β-actin can be used as the internal reference. A real-time quantitative PCR is carried out to detect methylation-specific amplification reaction of the gene markers. The commercially available fully methylated DNA can be used as a positive control. Because of the high ratio of unmethylated background DNA simultaneously isolated from blood or body fluids, measurement of unmethylated DNA amplification of the gene markers may not be necessary. A methylation index (MI) for each gene marker or total MI for the panel of gene markers assayed can be calculated based on the CT value of amplification reaction, according to following equation:

$$MI = \frac{{2}^{- ( G sampleCT - G control CT )} / {2}^{- ( G control CT - G control CT )}}{{2}^{- ( R sample CT - R control CT )} / {2}^{- ( R control CT - R control CT )}} \times 100$$

Here, G represents genes of interest and R represents the internal reference. Number of MI may vary, depending on the ratio of methylated DNA, from 0 to thousands. Based on the degree of MI, number of the methylated gene markers, cancer status such as cancer free, pre-cancer (high risk), or early cancer can be determined. The cutoff points for discriminating among non-cancer, pre-cancer, and early cancer could be established through clinical trial of large number of blood and body fluid samples.

If the detection of a panel of methylated marker genes in the blood sample of the subject is clearly shown to be cancerous status, then another panel of marker genes will be employed to localize cancer or classify the type of cancer detected. It could be achieved by identifying tissue origination of methylated DNA. According to the method of this invention, the tissue origin of methylated DNA could be clarified by using a panel of tissue- or cell-specific marker genes. These genes include but are not limited to cytokeratin 7 (CK7), cytokeratin 19 (CK 19), cytokeratin 20 (CK20), thyroid transcription factor-1 (TTF-1), surfactant protein A (SP-A), surfactant protein B (SP-B), surfactant protein D (SP-D), napsin A, small breast epithelial mucin (SBEM), mammaglobin A, mammaglobin B, prostate specific antigen (PSA), NK3 transcription factor related, locus 1 (NKX3-1), prostate cancer antigen 3 (PCA-3), Ebstein-Barr virus (EBV), methionine adenosyltransferase 1A (MAT-1A), methionine adenosyltransferase 2A (MAT-2A), alpha fetal protein (AFP), and pancreas duodenum homeobox-1 (PDX-1). According to the method of this invention, a panel of mathylation-based tissue- or cell specific gene markers selected from these genes comprise of CK7, CK20, TIF-1, mammaglobin A (MGBA), NKX3-1, EBV, MAT-1A, and PDX-1. The products of these genes are not normally contained in blood and bone marrow but these genes are expressed in different tissues of epithelial origin or as the exogenous nucleic acid (EBV).

CK7, and CK 20, as cytokeratin phenotyping, have exhibited that their expression at protein level and/or mRNA level is beneficial in identifying the origin of metastatic carcinoma of gastrointestinal tract (Tot et al: Eur J of Cancer, 38: 758-763, 2002). CK7 is mainly expressed in several tissues of epithelial origin, which include stomach, military, pancreas, breast ductal, breast lobular, endometrium, ovarium, and lung. CK7 is less expression in colon, prostate and kidney epithelium. CK20 is mainly expressed in colon, stomach, and biliary but barely expressed in breast, ovary, lung, prostate, and kidney. TIF-1 is a nuclear transcription protein and plays a role in transcriptional activation during embryogenesis in the thyroid and respiratory epithelium. TIF-1 is consistently expressed in all pulmonary epithelial cells during early embryogenesis and exclusively expressed in thyroid and lung carcinomas and is used for identifying the lung origin of metastatic carcinoma (Fabbro et al: Eur J of Cancer 32A, 512-517, 1996). MGBA expression has been detected only in the mammary gland (Watson et al: Cancer Res, 56, 860-865, 1996). NKX3-1 is an androgen-regulated gene and its expression is largely specific to the prostate epithelium (Voeller et al: Cancer Res, 57, 4455-4459, 1997). EBV infection is closely associated with nasopharyngeal carcinoma (Chan et al: Cancer Bio 12, 489-496, 2002). The level of EBV DNA in blood has been shown to be helpful in the diagnosis of nasopharyngeal carcinoma. MAT-1A encodes for methionine adenosyltransferase and is expressed only in the adult liver (Torresl et al: FASEB. 14, 95-102, 2000). PDX-1 is crucial for pancreatic organogenesis and is specifically detected in embryonic pancreas and is expressed in pancreatic duct cells that have the potential to differentiate into islets (Offield et al: Development, 983-995, 1996).

According to the method of this invention, methylation-based detection of these gene markers involves to analyze the unmethylation of the CpG islands in the promoter/exon1 region. Expression of these gene markers at RNA level in blood with exception of EBV was reported in prior art. Gene expression at RNA level is entirely or partially regulated by unmethylation status of promoter/exon1. Further, the tissue- or cell specific expression of a gene often involves to unmethylation of the promoter/exon1 region of a gene in the specific tissue or cells through epigenetic regulation. Thus analysis of methylation or unmethylation status in the promoter/exon1 regions of these gene markers could lead to know if these gene markers are expressed. In another word, the detected unmethylation of the promoter/exon1 regions of these gene markers could represent or reflect the expression of these gene markers at RNA level. According to the method of this invention, the advantage of unmethylation-based detection of tissue- or cell-specific gene markers is that only circulating DNA is required, which enables a small quantity of blood sample to be sufficient for an assay, whereas a large quantity of blood sample is required by using circulating RNA. Also DNA as the analyzed material is much more stable and consistent than RNA. Further, when an assay of early cancer discrimination is carried out, use of DNA could save time and reagents since same analyzed material and same assay protocol used for general cancer detection is utilized. In the promoters or exon1 regions of these tissue- or cell-specific gene markers, CpG islands or critical CpG sites were identified. For instance, one CpG island containing 12 CpGs is mapped to the 5′-UTR region of CK7 gene. Two CpG sites in the exon1 region of MAT1A were found to be unmethylated in liver but methylated in other tissues.

According to the method of this invention, DNA from plasma/serum or body fluids is chemically modified and used for unmethylation-based assay. An assay for unmethylation of these tissue- or cell-specific gene markers may include but are not limited to MS-PCR, Primer extension, COBRA, MethyLight, ConLight-MSP, McMSP, MSP/DHPLC, SNuPE, HM-MethyLight, strand displacement assay, ligase chain reaction, rolling circle amplification, loop-mediated amplification, Oligonucleotide microarrays, Bisulfite sequencing and PyroMeth. However, a suitable assay for routine clinical use should be rapid, cost-effective enough with high sensitivity and specificity. According to this invention, the assay for unmethylation of these tissue- or cell-specific gene markers is related to multiplex real-time gene methylation amplification. According to this method, the CpG-rich regions in promoter or exon 1 of a gene marker can be amplified using at least two primers and a probe which are complementary to a demethylated DNA modified with bisulfite. The primers are specific to demethylated DNA of interest and are able to initiate synthesis of a primer extension product. A sequence-specific probe is able to hybridize to the target between forward and reverse primer sites. The probe is labeled with a fluorescent dye at 5′ portion. The fluorescent dyes may include but are not limited to FAM, TET, Texas-red, cy3, cy5, JOE, HEX, MAX, ROX, TAMRA and VIC. The probe is quenched with a black hole quencher and conjugated with MGB protein at its 3′ portion. The MGB is able to fold into minor groove of DNA duplex created between the target sequence and probe. Thus the conjugation of MGB to the probe stabilizes annealing, allowing increased target discrimination, greater precision and consistence between individual assays. The probe used in the method of this invention can be also a Locked Nucleic Acid (LNA) probe which contains nucleosides having a methylene bridge that connects the 2′-oxygen of ribose with the 4′-carbon of the ribose ring. The LNA probe is also labeled with a fluorescent dye at 5′ portion and quenched with a black hole quencher at its 3′ portion. The LNA fluorescent probe may also increase thermal stability and hybridization specificity. Two or more gene markers can be multiplexed by labeling each probe with different dye in a single amplification reaction to save time and reagents. A housekeeping gene such as b-actin can be used as internal reference. A real-time quantitative PCR is carried out to detect methylation-specific amplification reaction of gene markers. A positive control can be set up by using cloned DNA fragments or preferably synthesized universal unmethylated DNA. The cloned DNA fragment contains the same sequence to be amplified in a gene marker and should be 200-600 by length. The universal unmethylated control DNA may be synthesized by whole genome amplification with phage29 DNA polymerase or with Bst DNA polymerase large fragment. The index (UMI) for each gene marker can be calculated based on the CT value of amplification reaction, according to following equation:

$$UMI = \frac{{2}^{- ( G sampleCT - G control CT )} / {2}^{- ( G control CT - G control CT )}}{{2}^{- ( R sample CT - R control CT )} / {2}^{- ( R control CT - R control CT )}} \times 100$$

Here, G represents genes of interest and R represents the internal reference. Number of UMI may vary, depending on ratio of demethylated DNA, from 0 to thousands. The cutoff points for identifying and discriminating cancer types could be established through clinical trial of large number of blood and body fluid samples.

According to the method of this invention, once cancerous status are suggested by a panel of methylated gene markers in the blood sample of the subject, the panel of tissue- or cell-specific gene markers are capable of identifying and discriminating the tissue source of cancer through unmethylation patterns of these combined gene markers. The unmethylation patterns of these gene markers will further lead to classify cancer types which may at least include lung, breast, ovarian, liver, stomach, colon, pancreatic, prostatic and nasopharyngeal cancer. For example, a lung cancer is suspected when the panel of tissue- or cell-specific gene markers presents the following unmethylation pattern: CK7 (+), CK20 (−), TTF-1 (+), NKX3-1 (+/−), MGBA (−), EBV (−), MAT1-A(−), PDX-1(−). Here (+) represents unmethylation and (−) represents methylation. The unmethylation patterns for indicating different types of cancer are listed in table 2.

TABLE 2
identification of cancer type based on unmethylation patterns of tissue-or cell-
specific gene markers

| | CK7 | CK20 | TTF-1 | NKX3-1 | MGBA | EBV | MAT1-A | PDX-1 |
|---|---|---|---|---|---|---|---|---|
| Lung | + | − | + | − | − | − | − | − |
| breast: | + | − | − | +/− | + | − | − | +/− |
| ovarian: | + | + | − | − | + | − | − | − |
| liver: | + | +/− | − | − | − | − | + | − |
| colon: | − | + | − | − | − | − | − | +/− |
| stomach | + | + | − | − | − | − | − | − |
| pancreas | + | + | − | − | − | − | − | + |
| prostate: | − | − | − | + | − | − | − | +/− |
| nasopharyngeal | +/− | − | − | − | − | + | − | − |

Some of cancer types identified using the panel of tissue- or cell-specific gene markers based on the method of this invention could be confirmed by a methylation-based assay using a sample collected directly from the tumor located sites. These samples, depending on cancer types, could be collected from tissue fluid aspirate, tissue swabs or lavage, local organ or tissue secretion, sputum, urine and stool. For example, if the lung cancer is identified, sputum or bronchoalveolar lavage may be collected for examining methylation status of the panel of cancer-related marker genes selected according the method of this invention. Similarly, the identified colon cancer, prostate cancer, breast cancer and nasopharyngeal cancer can be confirmed by examining methylation status of above marker genes using stool, prostate fluid, breast aspirate and nasal swab/wash, respectively. Conventional diagnostic tests can be further performed to confirm if a cancer mass exists at the time that methylation markers are detected in a biological sample of a subject. These conventional diagnostic tests may include chest X-rays, CT scanning, MRI, endoscopic examination, colorectal examination, imaging such as PET scanning and barium imaging. Even if no cancer mass is found, the conventional diagnostic tests may be still required to perform, depending on the positive degree of methylation-based assays, at the intervals from 3 to 12 months.

According to the method of this invention, a kit for detecting general cancer through examining mathylation status of a panel of cancer-related marker genes can be made. This kit includes: a) a lysis buffer system comprising various salts, detergents and a protein degrading enzyme; b) a DNA capture system comprising an apparatus with a pre-inserted solid matrix and a binding buffer; c) a DNA modification system comprising modification buffer and an apparatus with a pre-inserted solid matrix; d) methylation-specific DNA amplification system comprising a set of primer pairs, a set of oligonucleotide probes labeled with different dyes, DNA polymerase and amplification reaction buffer; e) an instruction for conducting an assay according to the method of this invention. A kit for identifying cancer types through examining unmethylation patterns of a panel of tissue- or cell-specific gene markers can be also made. The kit includes: a) a lysis buffer system comprising various salts, detergents and a protein degrading enzyme; b) a DNA capture system comprising an apparatus with a pre-inserted solid matrix and a binding buffer; c) a DNA modification system comprising modification buffer and an apparatus with a pre-inserted solid matrix; d) a unmethylation-specific DNA amplification system comprising a set of primer pairs, a set of oligonucleotide probes labeled with different dyes, DNA polymerase and amplification reaction buffer; e) an instruction for conducting an assay according to the method of this invention.

An advantage of the method of this invention is that a cancer can be detected through the multiplexed methylated gene marker (tumor-specific) amplification by using only blood or body fluids. Another advantage is that a special type of cancer can be identified through the multiplexed demethylated gene marker (tissue or cell-specific) amplification once a cancer is detected. Another advantage is that a high analytic sensitivity and specificity for cancer detection can be achieved through a specific designed and MGB conjugated probes. Another advantage is that the tumor DNA entry into circulation can be discriminated from circulating background DNA through use of tissue or cell-specific gene markers. The further advantage of the method of this invention is that a complete assay for cancer detection or identification (from blood collection to data completion) can be finished rapidly (within 4 hours)

The method of this invention can be used for detecting the early cancer; screening cancer risk, identifying special type of the cancer after cancer is detected, monitoring the cancer progress or prognosis, and evaluating the efficacy of cancer treatment such as chemotherapy, radiation, biotherapy and surgical operation.

The method of this invention for detecting early cancer or pre-cancer using blood and body fluids is further illustrated in the following examples:

### Example 1

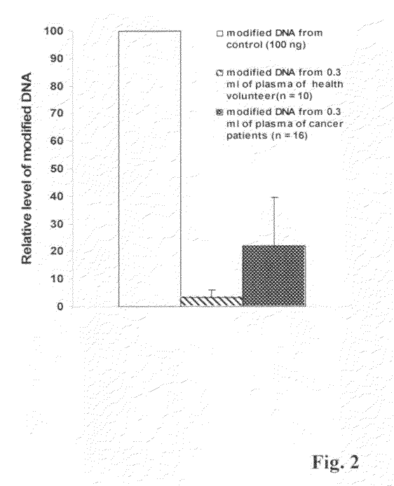

This experiment was carried out to show the isolation and modification of DNA from plasma by using the method of this invention. 1 ml of blood from health volunteers or cancer patients was collected using EDTA coated plasma collection tube. Blood sample was gently inverted several times and centrifuged for 10-20 min at 2000-3000 rpm. Upper plasma layer was carefully collected using a disposable transfer pipette. About 300 μl of plasma were then added to an equal volume of lysis buffer, which comprises a solution containing 20 mM Tris-HCl, 10 mM EDTA, 0.5% triton 100-X, 50 mM KCl, and 2.5 M NaCl with pH 9.0 and 0.025% of proteinase K (W/V). The mixture was incubated for 10 min at 65° C. and DNA was then precipitated by adding 0.5 volume of 100% isopropanol followed by centrifugation or captured with a column pre-inserted with a silica membrane or a silica filter. Isolated DNA was denatured with 0.2 M NaOH. As a comparison, commercial available human DNA (100 ng) was used as the control. Denatured DNA was then treated with a modification solution for 90 min at 65° C. The modification solution comprises 3.2 M of Na₂S₂O₅, 500 mM of KCl and 0.2 mM EDTA. The solution containing the modified DNA was mixed with DNA binding buffer comprising non-chaotropic salts and added into a column apparatus with inserted DNA capture filter. The mixed solution passed through the column in a receiver tube by centrifugation. Modified DNA was desulphorated and eluted from the DNA capture filter. The amount of modified DNA was examined by real-time quantitative PCR. Relative level of isolated DNA from plasma is calculated by using the equation: ½^{|ΔCt|}×100%. A pair of primers and a probe designed to amplify both methylated and unmethylated alleles of b-actin were used to quantify DNA. Primer sequences of β-actin are: forward GGAGGTAGGGAGTATATAGGT (SEQ ID NO. 1) and reverse CCAACACACAATAACAAACA (SEQ ID NO. 2). The probe sequence of β-actin is: 6-FAM-TGATGGAGGAGGTTTAG-BHQ MGB (SEQ ID NO. 3). As shown in FIG. 2, the level of modified DNA measured from 300 ul of plasma, depending on samples, ranged from 0.5% to 38% of that obtained from 100 ng of control DNA.

### Example 2

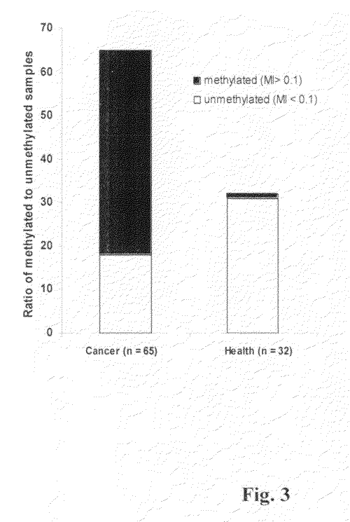

This experiment was carried out to show that DNA methylation analysis of a panel of marker genes using multiplexed real-time PCR based on this invention. 32 health volunteers and 62 patients with different cancer types were enrolled in this testing. The cancer types include colon-rectal cancer (20), hepatoma (14), stomach cancer (10), breast cancer (9), and esophagus cancer (9). All of the patients were confirmed pathologically and not treated before testing. 1 ml of blood was collected using EDTA coated plasma collection tube. DNA was isolated and modified as described in example 1. A real-time quantitative PCR was carried out to detect methylation-specific amplification reaction of gene markers. A panel of marker genes was comprised of p16, RASSF1A, APC, MGMT, hMLH1, GSTP1 and CDH-13. The primer and probe sequences for marker gene detection are listed below. In all cases, the first primer listed is the forward PCR primer, the second is the probe, and the third is the reverse PCR primer. P16 methylated: 5′-GTATGGAGTTTTCGGTTGA-3′ (SEQ NO. 4), 5′-TTACGGTCGCGGTT-3′ (SEQ NO. 5), 5′-ACCCACCTAAATCGACCT-3′ (SEQ NO. 6); RASSF1A methylated: 5′-CGTTTGTTAGCGTTTAAAG-3′(SEQ NO. 7), 5′-CGAAGTACGGGTTTAAT-3′ (SEQ N0.8), 5′-CCAACGAATACCAACTCC-3′ (SEQ NO. 9); APC methylated: 5′-TTGTGTAATTCGTTGGATG-3′ (SEQ NO. 10), 5′-AGTTCGTCGATTGGTT-3′ (SEQ NO. 11), 5′-CATATCGATCACGTACGC-3′ (SEQ NO. 12); MGMT methylated: 5′-TTCGGATATGTTGGGATAG-3′ (SEQ NO. 13), 5′-TTTCGACGTTCGTAGG-3′ (SEQ NO. 14), 5′-AACGACCCAAACACTCAC-3′ (SEQ NO. 15); hMLH1 methylated: 5′-TCGTTGAGTATTTAGACGTTT-3′ (SEQ NO. 16). 5′-TGTCGTTCGTGGTAGG-3′ (SEQ NO. 17), 5′-GCGATACGATTCACCACT-3′ (SEQ NO. 18); GSTP1 methylated: 5′-TATAAGGTTCGGAGGTCG-3′ (SEQ NO. 19), 5′-TTGGAGTTTCGTCGTC-3′ (SEQ NO. 20), 5′-CGCGCGTACTCACTAATA-3′ (SEQ NO. 21); CDH-13 methylated: 5′-TGTTTAGTGTAGTCGCGTGT-3′ (SEQ NO. 22), 5′-GAAAACGTCGTCGG-3′ (SEQ NO. 23), 5′-ACAAAACGAACGAAATTCTC-3′ (SEQ NO. 24). The 13-actin gene was used as an internal reference. The primers are specific to methylated DNA of interest. The sequence-specific probes were labeled with either FAM or TET. The probe was quenched with a black hole quencher and conjugated with a minor groove binder (MGB) protein at its 3′ portion. Two or more gene markers were multiplexed in a single amplification reaction tube. The commercial available fully methylated DNA was used as a positive control. 20 μl of PCR mixture was prepared by addition of the modified DNA, Taq enzyme, PCR buffer, 2 primer sets and 2 probes: one was labeled with FAM and another with TET. The PCR reactions were carried out with the following temperature settings: 1 cycle of 95 degree.C. for 4 minutes, 50 cycles of 94 degree.C. for 30 seconds, 55 degree.C. for 30 seconds and 72 degree.C. for 30 seconds. The gene methylation intensity (MI) was calculated by dividing the marker gene: b-actin ratio of a sample by the marker gene: b-actin ratio of the positive control. The total MI is the sum of individual MI for each marker gene. Among 65 cancer samples, 47 had the methylation alteration in at least one of marker genes and total MI was greater 0.1. Among 32 health individuals, only one was found to have methylation changes in the tested marker genes (FIG. 3)

### Example 3

This experiment was carried out to identify and discriminate one cancer type from others in a group of cancer types through unmethylation patterns of a panel of tissue- or cell-specific gene markers. Blood collection, DNA isolation and modification were carried out as described in example 1. A real-time quantitative PCR is carried out to detect methylation-specific amplification reaction of gene markers. A panel of marker genes is comprised of CK7, CK20, TTF-1, NKX3-1, MGBA, EBV, MAT1-A, PDX-1. The primer and probe sequences for marker gene detection are listed below. In all cases, the first primer listed is the forward PCR primer, the second is the probe, and the third is the reverse PCR primer. CK7 demethylated: 5′-TAGAGAAAGGTGGTTTGTGG-3′ (SEQ NO. 25), 5′-TGGATAAAAGGTGTGGA-3′ (SEQ NO. 26), 5′-AACACACACTCACTAACCTCA-3′ (SEQ NO. 27); CK20 demethylated: 5′-GGTATGTAGTGTTTTGGGATG-3′(SEQ NO. 28), 6 5′-AGGTTGGGGTATTTGTA-3′(SEQ N0.29), 5′-AACAAATCCCCACCACCT-3′ (SEQ NO. 30); TIF-1 demethylated: 5′-TATGTTTTGTTTTTGGTGG-3′ (SEQ NO. 31), 5′-ATTTGGTGTTGGGTTA-3′ (SEQ NO. 32), 5′-AAAACTCAAAAACTACCTCA-3′ (SEQ NO. 33); NKX3-1 demethylated: 5′-GGTTGTTGGGATGTTTAGG-3′ (SEQ NO. 34), 5′-TGGGGAGGTGAAAGT-3′ (SEQ NO. 35), 5′-CACACCATCCCACAAAATA-3′ (SEQ NO. 36); MGBA demethylated: 5′-GTGGTTTTTTTGATTTTTG-3′ (SEQ NO. 37), 5′-TGTGATTGAATATTGATAG-3′(SEQ N0.38), 5′-AACTCACCTACATAACAATACT-3′ (SEQ NO. 39); EBV demethylated: 5′-TTATTTTTTTTGGTTGGTGG-3′ (SEQ NO. 40), 5′-TGTTTCGTGTTTTATGG-3′(SEQ N0.41), 5′-AACCAAACCAATAACAATCACA-3′ (SEQ NO. 42); MAT1-A demethylated: 5′-ATATATAGGAGTTGTTTAGGAG-3′ (SEQ NO. 43), 5′-GGAGGGATATATTTTG-3′(SEQ NO. 44), 5′-CCTAACTACCTATAAACACAT-3′ (SEQ NO. 45); PDX-1 demethylated: 5′-GGTTGTAGTTATGAATGGTGA-3′ (SEQ NO. 46), 5′-AGGATTTATGTGTGTTTTAG-3′ (SEQ NO. 47), 5′-TAAACTCCAACACCAAACCT-3′ (SEQ NO. 48). The β-actin gene is used as an internal reference. The primers are specific to unmethylated DNA markers of interest. Two or more gene markers were multiplexed in a single amplification reaction tube. The sequence-specific probes are labeled with either FAM or TET. The probe is quenched with a black hole quencher and conjugated with a minor groove binder (MGB) protein at its 3′ portion. The universal unmethylated DNA is used as the positive control. 20 ul of PCR mix is prepared by addition of the modified DNA, Taq enzyme, PCR buffer, 2 primer sets and 2 probes: one is labeled with FAM and another with TET. The PCR reactions are carried out with the following temperature settings: 1 cycle of 95 degree.C. for 4 minutes, 50 cycles of 94 degree.C. for 4 seconds, 55 degree.C. for 10 seconds and 72 degree.C. for 6 seconds. The gene unmethylation intensity was calculated by dividing the marker gene: β-actin ratio of a sample by the marker gene: β-actin ratio of the positive control. The cut-off point of UMI for each gene marker is set up as 1. Cancer type is discriminated based on the unmethylation patterns of tissue- or cell-specific gene markers as listed in Table 2.

### Example 4

This experiment was carried out to confirm the cancer type by a methylation-based assay using a sample collected directly from the tumor located sites. The samples are collected directly from tumor located sites based on susceptible cancer type identified by the unmethylation patterns of tissue- or cell-specific gene markers. For tissue fluid aspirate, tissue swabs or lavage, local organ or tissue secretion and urine, the samples are collected into 15 ml conical tube and centrifuged at 2000 rpm for 10 min to pellet cells. Cells are washed with 10 ml of PBS and DNA is isolated and modified as described in example 1. Remove For sputum or mucoid samples, the commercial protocol such as DTT-sputolysin method can be used to collect cells. A real-time quantitative PCR is carried out to detect methylation-specific amplification reaction of gene markers. A panel of marker genes is comprised of p16, RASSF1A, APC, MGMT, hMLH1, GSTP1, CDH-13. One of following marker genes, depending on the source of body fluids, can be also added: CK7, CK20, TTF-1, NKX3-1, MGBA, EBV, MAT1-A and PDX-1. The primer and probe sequences for these marker genes were listed in example 2 and 3, respectively. The β-actin gene was used as an internal reference. The primers are specific to methylated DNA of interest. The sequence-specific probes were labeled with either FAM or TET. The probe was quenched with a black hole quencher and conjugated with a minor groove binder (MGB) protein at its 3′ portion. Two or more gene markers are multiplexed in a single amplification reaction tube. The commercial available fully methylated DNA is used as a positive control. 20 μl of PCR mix are prepared by addition of the modified DNA, Taq enzyme, PCR buffer, 2 primer sets and 2 probes: one is labeled with FAM and another with TET. The PCR reactions are carried out with the following temperature settings: 1 cycle of 95 degree.C. for 4 minutes, 50 cycles of 94 degree.C. for 4 seconds, 55 degree.C. for 10 seconds and 72 degree.C. for 6 seconds. The gene methylation intensity is calculated by dividing the marker gene: β-actin ratio of a sample by the marker gene: β-actin ratio of the positive control. The confirmation of special cancer type can be made by detected methylation alterations of at least one of marker genes.

What is claimed is:

1. A method for identifying and distinguishing at least one type from others in a group of cancer types at their early stage by using blood or plasma/serum from human comprising:
a) obtaining and bisulfite treating DNA in said sample;
b) detecting the amount or presence of a group of methylated DNA markers followed by detecting the amount or presence of a group of unmethylated DNA markers using quantitative PCR amplification with a group of primers and a group of probes, wherein the detected methylated DNA markers are indicative of the presence of cancer, and the detected unmethylated DNA markers are indicative of tissue, cell, or organ-specific origination of said cancer, thereby identifying and distinguishing the type of said cancer.

2. The method of claim 1 wherein said a group of cancer types are comprised of lung cancer, breast cancer, ovarian cancer, colon cancer, stomach cancer, pancreatic cancer, liver cancer, prostate cancer, thyroid cancer, and nasopharyngeal cancer.

3. The method of claim 1, wherein said a group of methylated DNA markers are selected from genes involved in tumor suppression, DNA repair, anti-proliferation and cell cycle regulation.

4. The method of claim 1, wherein said unmethylated DNA markers are selected from genes involved cell or tissue-specific origination, development and differentiation of said cancer.

5. The method of claim 1, wherein said group of methylated DNA markers is comprised of p16 (INK4A), RASSF1A, APC, MGMT, GSTP1, CDH-13 and MLH1.

6. The method of claim 1, where said group of unmethylated DNA markers is comprised of CK7, CK20, TTF-1, mammaglobin A, NKX3-1, EBV, MAT-2 and PAX-1.


SEQUENCE LISTING (48 sequences)

SEQ ID NO: 1 - DNA, 21 bp, Homo sapiens
  Source (344)...(363): Every C is replaced by T.
ggaggtagggagtatataggt

SEQ ID NO: 2 - DNA, 20 bp, Homo sapiens
  Source (434)(453): Complementary to the sequence in which every C is replaced by T.
ccaacacacaataacaaaca

SEQ ID NO: 3 - DNA, 17 bp, Homo sapiens
  Source (393)(409): Every C is replaced by T. 5 substitution with fluorescent reporter dye 6FAM; 3 substitution with black hole quencher.
tgatggaggaggtttag

SEQ ID NO: 4 - DNA, 19 bp, Homo sapiens
  Source (127)(145): Every C is replaced by T except C before G.
gtatggagttttcggttga

SEQ ID NO: 5 - DNA, 14 bp, Homo sapiens
  Source (154)(167): Every C is replaced by T except C before G. 5 substitution with fluorescent reporter dye 6FAM; 3 substitution with black hole quencher.
ttacggtcgcggtt

SEQ ID NO: 6 - DNA, 18 bp, Homo sapiens
  Source (243)(260): Complementary to the sequence in which every C is replaced by T except C before G.
acccacctaaatcgacct

SEQ ID NO: 7 - DNA, 19 bp, Homo sapiens
  Source (18040)(18058): Every C is replaced by T except C before G.
cgtttgttagcgtttaaag

SEQ ID NO: 8 - DNA, 17 bp, Homo sapiens
  Source (18063)(18079): Every C is replaced by T except C before G. 5 substitution with fluorescent reporter dye 6FAM; 3 substitution with black hole quencher.
cgaagtacgggtttaat

SEQ ID NO: 9 - DNA, 18 bp, Homo sapiens
  Source (18117)(18135): Complementary to the sequence in which every C is replaced by T except C before G.
acccacctaaatcgacct

SEQ ID NO: 10 - DNA, 19 bp, Homo sapiens
  Source 741)(760): Every C is replaced by T except C before G.
ttgtgtaattcgttggatg

SEQ ID NO: 11 - DNA, 16 bp, Homo sapiens
  Source (790)(805): Every C is replaced by T except C before G. 5 substitution with fluorescent reporter dye TET; 3 substitution with black hole quencher.
agttcgtcgattggtt

SEQ ID NO: 12 - DNA, 18 bp, Homo sapiens
  Source (813)(831): Complementary to the sequence in which every C is replaced by T except C before G.
catatcgatcacgtacgc

SEQ ID NO: 13 - DNA, 19 bp, Homo sapiens
  Source (1027)(1046): Every C is replaced by T except C before G.
ttcggatatgttgggatag

SEQ ID NO: 14 - DNA, 16 bp, Homo sapiens
  Source (1050)(1065): very C is replaced by T except C before G. 5 substitution with fluorescent reporter dye TET; 3 substitution with black hole quencher.
tttcgacgttcgtagg

SEQ ID NO: 15 - DNA, 18 bp, Homo sapiens
  Source (1115)(1132): Complementary to the sequence in which every C is replaced by T except C before G.
aacgacccaaacactcac

SEQ ID NO: 16 - DNA, 21 bp, Homo sapiens
  Source (74)(94): Every C is replaced by T except C before G.
tcgttgagtatttagacgttt

SEQ ID NO: 17 - DNA, 16 bp, Homo sapiens
  Source (118)(133): Every C is replaced by T except C before G. 5 substitution with fluorescent reporter dye TET; 3 substitution with black hole quencher.
tgtcgttcgtggtagg

SEQ ID NO: 18 - DNA, 18 bp, Homo sapiens
  Source (158)(175): Complementary to the sequence in which every C is replaced by T except C before G.
gcgatacgattcaccact

SEQ ID NO: 19 - DNA, 18 bp, Homo sapiens
  Source (1856)(1873): Every C is replaced by T except C before G.
tataaggttcggaggtcg

SEQ ID NO: 20 - DNA, 16 bp, Homo sapiens
  Source (1885)(1902): Every C is replaced by T except C before G. 5 substitution with fluorescent reporter dye 6FAM; 3 substitution with black hole quencher.
ttggagtttcgtcgtc

SEQ ID NO: 21 - DNA, 18 bp, Homo sapiens
  Source (1912)(1929): Complementary to the sequence in which every C is replaced by T except C before G.
cgcgcgtactcactaata

SEQ ID NO: 22 - DNA, 20 bp, Homo sapiens
  Source (1610)(1629): Every C is replaced by T except C before G.
tgtttagtgtagtcgcgtgt

SEQ ID NO: 23 - DNA, 14 bp, Homo sapiens
  Source (1635)(1648): Every C is replaced by T except C before G. 5 substitution with fluorescent reporter dye TET; 3 substitution with black hole quencher.
gaaaacgtcgtcgg

SEQ ID NO: 24 - DNA, 20 bp, Homo sapiens
  Source (1696)(1677): Complementary to the sequence in which every C is replaced by T except C before G.
acaaaacgaacgaaattctc

SEQ ID NO: 25 - DNA, 20 bp, Homo sapiens
  Source (3882)(3901): Every C is replaced by T.
tagagaaaggtggtttgtgg

SEQ ID NO: 26 - DNA, 17 bp, Homo sapiens
  Source (3945)(3961): Every C is replaced by T. 5 substitution with fluorescent reporter dye 6FAM; 3 substitution with black hole quencher.
tggataaaaggtgtgga

SEQ ID NO: 27 - DNA, 21 bp, Homo sapiens
  Source (3969)(3989): Complementary to the sequence in which every C is replaced by T. PUBLICATION INFORMATION
aacacacactcactaacctca

SEQ ID NO: 28 - DNA, 21 bp, Homo sapiens
  Source (4963)(4983): Every C is replaced by T.
ggtatgtagtgttttgggatg

SEQ ID NO: 29 - DNA, 17 bp, Homo sapiens
  Source (5010)(5026): Every C is replaced by T. 5 substitution with fluorescent reporter dye 6FAM; 3 substitution with black hole quencher.
aggttggggtatttgta

SEQ ID NO: 30 - DNA, 18 bp, Homo sapiens
  Source (5070)(5087): Complementary to the sequence in which every C is replaced by T. PUBLICATION INFORMATION
aacaaatccccaccacct

SEQ ID NO: 31 - DNA, 19 bp, Homo sapiens
  Source (89)(107): Every C is replaced by T.
tatgttttgtttttggtgg

SEQ ID NO: 32 - DNA, 16 bp, Homo sapiens
  Source (117)(132): Every C is replaced by T. 5 substitution with fluorescent reporter dye 6FAM; 3 substitution with black hole quencher.
atttggtgttgggtta

SEQ ID NO: 33 - DNA, 20 bp, Homo sapiens
  Source (152)(161): Complementary to the sequence in which every C is replaced by T. PUBLICATION INFORMATION
aaaactcaaaaactacctca

SEQ ID NO: 34 - DNA, 19 bp, Homo sapiens
  Source (39)(57): Every C is replaced by T.
ggttgttgggatgtttagg

SEQ ID NO: 35 - DNA, 15 bp, Homo sapiens
  Source (75)(89): Every C is replaced by T. 5 substitution with fluorescent reporter dye 6FAM; 3 substitution with black hole quencher.
tggggaggtgaaagt

SEQ ID NO: 36 - DNA, 19 bp, Homo sapiens
  Source (147)(165): Complementary to the sequence in which every C is replaced by T. PUBLICATION INFORMATION
cacaccatcccacaaaata

SEQ ID NO: 37 - DNA, 19 bp, Homo sapiens
  Source (1000)(1018): Every C is replaced by T.
gtggtttttttgatttttg

SEQ ID NO: 38 - DNA, 19 bp, Homo sapiens
  Source (1023)(1041): Every C is replaced by T. 5 substitution with fluorescent reporter dye 6FAM; 3 substitution with black hole quencher.
tgtgattgaatattgatag

SEQ ID NO: 39 - DNA, 22 bp, Homo sapiens
  Source (1096)(1117): Complementary to the sequence in which every C is replaced by T. PUBLICATION INFORMATION
aactcacctacataacaatact

SEQ ID NO: 40 - DNA, 20 bp, Homo sapiens
  Source (94)(113): Every C is replaced by T.
ttattttttttggttggtgg

SEQ ID NO: 41 - DNA, 17 bp, Homo sapiens
  Source (121)(137): Every C is replaced by T. 5 substitution with fluorescent reporter dye 6FAM; 3 substitution with black hole quencher.
tgtttcgtgttttatgg

SEQ ID NO: 42 - DNA, 22 bp, Homo sapiens
  Source (124)(175): Complementary to the sequence in which every C is replaced by T. PUBLICATION INFORMATION
aaccaaaccaataacaatcaca

SEQ ID NO: 43 - DNA, 22 bp, Homo sapiens
  Source (2073)(2094): Every C is replaced by T.
atatataggagttgtttaggag

SEQ ID NO: 44 - DNA, 16 bp, Homo sapiens
  Source (2115)(2131): Every C is replaced by T. 5 substitution with fluorescent reporter dye 6FAM; 3 substitution with black hole quencher.
ggagggatatattttg

SEQ ID NO: 45 - DNA, 21 bp, Homo sapiens
  Source (2175)(2195): Complementary to the sequence in which every C is replaced by T. PUBLICATION INFORMATION
cctaactacctataaacacat

SEQ ID NO: 46 - DNA, 21 bp, Homo sapiens
  Source (273)(293): Every C is replaced by T.
ggttgtagttatgaatggtga

SEQ ID NO: 47 - DNA, 20 bp, Homo sapiens
  Source (326)(385): Every C is replaced by T. 5 substitution with fluorescent reporter dye 6FAM; 3 substitution with black hole quencher.
aggatttatgtgtgttttag

SEQ ID NO: 48 - DNA, 20 bp, Homo sapiens
  Source (348)(367): Complementary to the sequence in which every C is replaced by T. PUBLICATION INFORMATION
taaactccaacaccaaacct